(19) United States
(12) Patent Application Publication
(10) Pub. No.: US 2025/0364125 A1
(43) Pub. Date: Nov. 27, 2025

(54) LOGISTICS MANAGEMENT METHOD FOR A SURGICAL SERVICE AREA

(71) Applicant: Medvision SAS, Medellin (CO)

(72) Inventor: Andres Felipe Bustamante Obando, Medellin (CO)

(21) Appl. No.: 19/219,877

(22) Filed: May 27, 2025

(30) Foreign Application Priority Data
May 27, 2024 (CO) NC2024/0006733

(51) Int. Cl. G16H 40/20

(52) U.S. Cl. CPC G16H 40/20

(57) ABSTRACT

The present development relates to a method for managing the logistics of a customer service area, such as an operating room, which improves the process of scheduling procedures and the management of resources assigned to the allocation of a customer service area (0). This is achieved through the use of technological surveillance tools. In particular, the disclosed method takes into consideration the use of electronic identification elements, such as, for example, a radio frequency system (RFID tag), a real-time location system (RTLS tag), or a Bluetooth beacon system, linked to a user or assistant, a supply, or equipment. This information allows to establish a status of the service provision and to carry out a statistical analysis that allows to obtain patterns in terms of to the interactions between staff, supplies and equipment in relation to a service area, historically.

### TECHNICAL FIELD

This disclosure relates to methods applied to scheduling and managing service spaces and resources in facilities dedicated to providing services. Specifically, this disclosure relates to logistics management methods for service spaces through procedure scheduling and resource management.

### SPECIFICATION OF THE PRIOR ART

The implementation of technologies and methodologies to optimize efficiency and improve care and space management in facilities dedicated to providing services, especially in critical environments such as healthcare facilities, has led to significant progress in allocation processes and utilization indicators related to user care.

Specifically, in the area of surgical service area management, scheduling and scheduling systems have been developed that use algorithms to improve resource management. In this way, the incorporation of real-time tracking systems allows healthcare teams to monitor the progress of surgeries, optimizing time and ensuring better coordination among professionals. Furthermore, the implementation of information technologies has enabled greater efficiency in the management of medical supply inventories, reducing the risk of shortages or excesses.

For example, a surgical procedure requires a complex care circuit, involving coordination between different professionals and the adjustment of scheduled (theoretical) and actual time, which allows for an equitable distribution of the assigned resources.

According to the above, surgical suites consume a significant amount of resources, which is why there are problems in this context related to waiting lists for the required surgical activity, for example, elective surgeries. In this case, the care process includes stages of patient and instrument preparation, anesthesia induction and termination, surgery, resuscitation, incorporation of process data into information systems, cleaning, etc. These stages involve coordinating operating room, professional, and patient schedules, and adjusting the scheduling of scheduled and utilized time to ensure appropriate resource allocation.

Scheduling operating rooms for elective surgeries depends on the demand for surgeries authorized by insurance companies (companies responsible for health promotion, health service provision, health resource management, and risk management) with which a health care center may have contracts. This scheduling is carried out by each institution according to the methods stipulated by each, but is optimized based on the analysis of the results of real-time scheduling using a workflow system.

Likewise, requests for supplies to the health center's warehouse area can come from a surgery scheduling area or from a hospitalization area. This creates a busy workflow related to scheduling and assigning spaces, such as operating rooms and recovery rooms.

The application of technologies such as the Internet of Things (IoT) has facilitated the creation of intelligent environments, where sensors and devices connected to data processing systems facilitate the monitoring of room occupancy. In this sense, patent documents GB2560506A and U.S. Pat. No. 11,315,678B2 refer to systems and methods aimed at scheduling and monitoring procedures in operating rooms that make use of intelligent environments.

In particular, GB2560506A discloses a computer-implemented resource allocation system and a computer-implemented method for allocating resources to events. The resource allocation system of GB2560506A is configured to manage a plurality of resource records, wherein each resource is a human resource or an equipment resource, and wherein said records further comprise information describing a location.

The method disclosed in GB2560506A indicates that the location of each resource can be determined with respect to the proximity of the resource to a location beacon, where, in case of the occurrence of an event, a notification can be transmitted to a communication device associated with the required resource. This reduces the time between reporting the occurrence of an event and deploying the resource.

For its part, document U.S. Pat. No. 11,315,678B2 discloses a method and system for developing an intelligent programming board for operating rooms in surgical centers and hospitals. The system disclosed in U.S. Pat. No. 11,315,678B2 comprises a scheduling server and a scheduling board.

The method described in U.S. Pat. No. 11,315,678B2 comprises the steps of monitoring the estimated location of a patient, monitoring the condition of a patient, displaying the estimated location of the patient on the scheduling board, displaying information about the condition of the patient on the scheduling board, and automatically updating the scheduling board as the location and condition of the patient change. Patient location information is estimated using an indoor location system.

According to the above, the state of the art discloses systems and methods that allow monitoring of patient status and the deployment of resources in service areas; however, no method is identified that allows scheduling that includes a projection of the use of supplies related to different procedures.

In this regard, Thijs Knoeff's paper, “Operating Room Scheduling,” describes the results of an evaluation of various scheduling approaches for an operating room scheduling system. In particular, Knoeff's paper highlights the benefits of using computerized tools, guided by the premise that surgeries of the same type tend to have similar logistical scheduling parameters.

For its part, patent document US20210043309A1 discloses a method for managing a hospital's inventory, said method comprising the steps of receiving user information, processing the user information, obtaining mapping information for medical procedures, processing said mapping information, and generating inventory forecast information and/or a medical procedure forecast from the collected historical information.

In particular, the inventory forecast disclosed in US20210043309A1 refers to the inventory of items required by a medical facility. The procedure forecast disclosed in US20210043309A1 refers to the number of medical procedures that will take place at the medical center. The user and mapping information referred to in US20210043309A1 is information that is entered into a processing unit by a user.

In short, logistics space management, especially in critical environments such as healthcare facilities, has evolved toward smarter and more efficient solutions thanks to the incorporation of advanced technologies and more precise approaches to planning and executing activities.

However, managing inventory and scheduling simultaneously for logistics planning in service spaces, including the integration of information systems and technologies, remains a challenge. Hospitals often have separate systems that don't communicate effectively with each other, making coordination and decision-making difficult. This is because the lack of real-time visibility into occupancy status and inventory levels can lead to interruptions and delays in service delivery. Furthermore, it prevents the generation of indicators of the use of these service spaces.

### BRIEF DESCRIPTION

This disclosure relates to a method for the logistical management of a service area. This method includes several stages related to scheduling a service, where, first, a computing unit obtains data on the requirement for providing a service in a service space, and availability data related to the resources that can be assigned to said service space. Subsequently, through a scheduling process, an assignment data is generated from the requirement data and the availability data.

Said allocation data includes space allocation data related to at least one service space; personnel allocation data related to at least one electronic identification element; supply allocation data related to at least one electronic identification element; and equipment allocation data related to at least one electronic identification element.

In particular, the computing unit is in communication with a data acquisition device. Said data acquisition device is related to at least one service space, where identification data is generated from an electronic identification element. The identification data includes personnel identification data, supply identification data, and equipment identification data.

This identification data, together with the assignment data, is processed in the computing unit through a verification process of minimum requirements to generate a registration data.

### BRIEF DESCRIPTION OF THE DRAWINGS

FIG. 1 shows a flow diagram of one embodiment of the present disclosure.

FIG. 2 shows a block diagram of one embodiment of the present disclosure, detailing the interaction with associated resources and devices.

FIG. 3 shows a flowchart of an embodiment of the present disclosure wherein a method-related statistical data is generated.

FIG. 4 shows a flowchart of an embodiment of the present disclosure wherein a requirement is generated, and where the availability data is modified through an inventory adjustment process.

FIG. 5 shows a block diagram of an embodiment of the present disclosure, detailing interaction with associated resources and devices, and including a display device.

### DETAILED DESCRIPTION

The present disclosure is directed to a method for the logistical management of a service area (2), in particular, a service area (2) associated with a care center. With reference to the above, this disclosure discloses a method that allows optimizing logistics programming in service areas in an integral manner, that is, a logistics programming that simultaneously includes the management of resources and service areas, where said resources can refer to an element (supplies and equipment), a service area (2), and human resources (personnel).

This is possible by linking information from each procedure through monitoring of supplies, equipment, personnel, and service users. In this sense, this disclosure allows for reducing wait times related to the preparation of a service space, generating alerts related to the use of said service spaces (proper use of the service space, completeness of required elements, staff entry, use of supplies), optimizing the allocation and use of resources for a service, and obtaining a statistical analysis and management indicators related to each space.

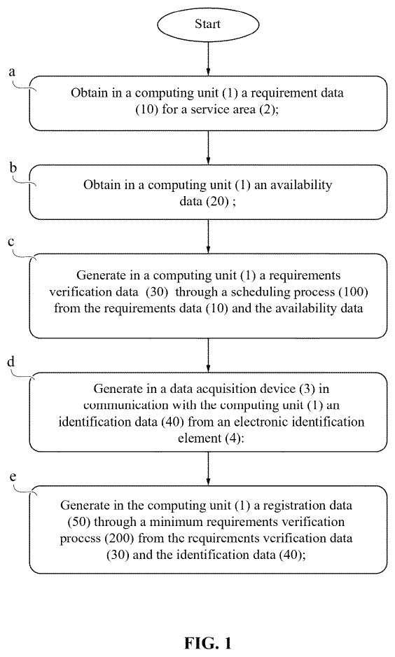

In accordance with the foregoing, and in relation to FIG. 1, one embodiment of the present disclosure relates to a method for the logistical management of a service space comprising the following steps:
a) obtain in a computing unit (1) a requirement data (10) for the attention space (2);
b) obtain in the computing unit (1) an availability data (20);
c) generate in the computing unit (1) a requirements verification data (30) through a scheduling process (100) from the requirements data (10) and the availability data (20), where the requirements verification data (30) comprises:
   a space allocation data (31) related to at least one service area (2);
   a personnel assignment data (32) related to at least one electronic identification element (4);
   an supply allocation data (33) related to at least one electronic identification element (4); and
   an equipment allocation data (34) related to at least one electronic identification element (4).
d) generating in a data acquisition device (3) in communication with the computing unit (1) an identification data (40) from an electronic identification element (4), wherein the identification data (40) comprises:
   a personal identification data (42);
   an supply identification data (43); and
   an equipment identification data (44);
e) generate in the computing unit (1) a registration data (50) through a minimum requirements verification process (200) from the requirements verification data (30) and the identification data (40);

In particular, the data acquisition device (3) is related to at least one attention space (2).

In this sense, according to this disclosure and FIG. 1, in step a) the requirements data (10) for the service space (2) is obtained in the computing unit (1). During said stage a), a person can use the computing unit (1) to generate the requirement data (10), for example, by using a Human Interface Device, or by using a data acquisition device. For example, said person may be a service center customer service staff member, who enters the data for a service request, for example, identification of the required service space (2), identification of the required staff, identification of the required resources, information on the required time of occupation of the service space, among others. This information forms a block of information that is entered into the computing unit (1), and that is necessary to generate the requirement data (10), where the block of information is a set of data that allows its manipulation by computational means.

In another embodiment of the present disclosure, during step a) a remote device may be linked to the computing unit (1) to generate the request data (10), for example, by using local communication networks, or by remote connections, in order to obtain the information necessary for generating the request data (10).

In this sense, the requirement data (10) can be generated locally or remotely, so that it is obtained in the computing unit (1) as a complete block of information. In particular, the requirement data (10) refers to the conditions necessary for the provision of a service, which are related to the need to occupy the service space (2) and the resources required to provide said service.

According to the above, the requirement data (10) allows to identify the conditions to select the resources necessary to provide a service in a service area (2). In this way, the requirement data (10) may include a required space data (11), a required personnel data (12), a required input data (13), and a required equipment data (14).

Optionally, the requirement data (10) may include a required time data (15), which allows the estimated occupation time for the provision of a service in the service space (2) to be specified, in accordance with a time standard established for the service provided.

Additionally, the requirement data (10) may include a required service data (16), which allows identifying a type of service within a broader set of services available at the service center.

In one embodiment of the present disclosure, the required time data (15) may be linked to the required personnel data (12) and to the required service data (16), that is, the required time data (15), which allows to specify the estimated occupation time for the provision of a service in the service space (2), may depend on the type of service and the person in charge of providing said service.

For its part, with reference to stage b), simultaneously or sequentially with stage a), the availability data (20) is obtained in the computing unit (1). The availability data (20) can be stored directly on the computing unit (1) or on a remote device.

Said availability data (20) is related to the capacity of the care center, said capacity has to do with an occupation that is recorded for each of the service areas (2) and with available resources that are associated with the care center.

In accordance with the above, the availability data (20) can be understood as information related to a service center, specifically with a service space (2) or with a resource, where said information allows to identify the projected occupation time of the service spaces (2) and the availability of the resources that can be assigned to fulfill the provision of a service.

In one embodiment of the present disclosure, the availability data (20) may be configured to be stored in a database.

In one embodiment of the present disclosure, the availability data (20) may be an inventory, or an electronic inventory document.

In this way, the availability data (20) may include available space data (21), available personnel data (22), available input data (23), and available equipment data (24).

In accordance with the above, in one embodiment of the present disclosure, the availability data (20) is generated in the computing unit (1) by performing a verification of available resources from the requirement data (10) and making a reservation of resources from said available resources. Such reservation may be made in accordance with the areas involved in providing a service in the service area (2).

According to the previous modality, stage b) may comprise the following sub-stages to obtain availability data (20) in the computing unit (1):
verify the availability of resources from the requirement data (10); and
record in the computing unit (1) an availability data (20) based on the availability of resources verified in the previous stage.

For example, in a modality in which the service area (2) is an operating room, the areas involved in providing the service may be: a surgery programming area, a warehouse area (supplies management), and a biomedical area (equipment management).

In a modality where the service area (2) is an operating room and where the surgery scheduling area makes a personnel reservation, the personnel of the surgery scheduling area can verify the availability of service personnel, including specialists and assistance personnel, according to the required personnel registered in the required personnel data (12), said information can be registered in the available personnel data (22).

Additionally, in a modality where the service area (2) is an operating room and where the surgery scheduling area makes a reservation for a service area (2), the staff of the surgery scheduling area can verify the availability of service areas (2), for example, operating rooms, according to the required staff recorded in the required space data (11), taking into account the nature and complexity of the procedure. Such information may be recorded in the available space data (21).

Thus, the verification and registration of resources during stage b) may comprise the following sub-stages:
verify the availability of service personnel based on the required personnel data (12);
record in the computing unit (1) a data of available personnel (22) from the availability of personnel;
verify the availability of service spaces (2) based on the required space data (11);
record in the computing unit (1) a data of available space (21) based on the availability of the service space (2);

In a modality where the service area (2) is an operating room and where the surgery scheduling area verifies the allocation of resources with a resource manager, the surgery scheduling area, the surgery scheduling area staff can perform a verification with an insurer.

Therefore, optionally, before the stage of verifying the availability of service personnel, a stage of verifying the allocation of resources with a resource manager can be performed.

In a modality where the service area (2) is an operating room and where the warehouse area verifies the availability of the supplies recorded in the required input data (13) and in the required service data (16), the warehouse area staff can verify the availability of the supplies recorded in the required input data (13), required for a service. Once the availability of these inputs is verified, said availability can be recorded to generate the available input data (23).

That is, the verification and registration of resources during stage b) may comprise the following sub-stages:
verify the availability of inputs based on the required input data (13) and the required service data (16);
record in the computing unit (1) an available input data (23) based on the availability of inputs.

In a modality where the service area (2) is an operating room and where the biomedical area verifies the availability of the equipment registered in the required equipment data (14). After verifying the availability of said equipment, said availability can be recorded to generate the available equipment data (24).

In one embodiment of this disclosure, biomedical personnel may perform equipment verification based on availability times. That is, the biomedical area staff can verify the availability of the equipment based on the data recorded in the required equipment data (14) and in the required time data (15).

Thus, the verification and registration of resources during stage b) may comprise the following sub-stages:
Check the availability of equipment based on the required equipment data (14) and at least one required time data (15)
record in the computing unit (1) a piece of available equipment data (2) based on the availability of equipment.

Optionally, in one embodiment of the present disclosure, the biomedical area personnel can update a resume of the equipment, that is, record its use, rotation and maintenance performed or pending, after generating the data of available equipment (24). That is, after the stage of verifying equipment availability, a stage can be carried out to update an equipment life sheet.

Likewise, in one embodiment of the present disclosure, there may be equipment that is needed in the same time range to be used in several service areas (2), for example, several operating rooms. According to the above, there may be more than one requirement data (10), therefore, more than one required time data (15) and more than one required equipment data (14), where said required time data (15) are equal and said required equipment data (14) are equal.

According to the above, the biomedical area staff can verify the number of rotations that said equipment can have, that is, the same equipment can be transferred a number of times by different services in different service areas (2). The above allows the same team to be assigned. In this way, the available equipment data (24), where said available equipment data (24) includes the number of rotations scheduled for a piece of equipment, can be assigned for the provision of more than one service during the same period of time.

After obtaining the requirement data (10) and the availability data (20), in step c) a requirement verification data (30) can be generated in the computing unit (1), based on the common characteristics between the requirement data (10) and the availability data (20) through the scheduling process (100). In particular, the requirement data (10) allows for the establishment of restrictions by means of which availability data (20) that meets the conditions for satisfying said restrictions is identified and related. According to the above, the requirements verification data (30) is configured from the availability data (20) and the requirement data (10) and allows the referential integrity of said relationship to be guaranteed.

The scheduling process (100) of step c) may be, for example, a relational process, performed manually, or it may be configured to be executed automatically in the computing unit (1).

For example, in one embodiment of the present disclosure, during step c), the scheduling process (100) for a service center with two service spaces (2A, 2B), is based on the requirement data (10) obtained in the computing unit (1) that refers to a service space required for 2:00 pm on Monday. This requirement data can be entered by a person into the computing unit (1). For its part, the availability data (20) is obtained simultaneously in the computing unit (12), which can be stored directly in the computing unit (1), and which indicates that one of the service spaces (2A) has availability between 8:00 am and 11:00 am on Monday, while the service space (2B) has availability between 12:00 pm and 5:00 pm on Monday. According to the above, during the scheduling process (100), carried out manually, a person can assign the service space (2B) for the provision of the required service, so the requirements verification data (30) for the previous example is related to the service space (2B) at 2:00 pm on Monday.

In this way, the requirements verification data (30) allows us to identify a service that will be provided in a service space (2). Said requirements verification data (30) includes information related to the assigned service space (2), in addition to being related to the resources corresponding to the provision of a service in said service space (2), where said resources are related to an electronic identification element (4). In this sense, the requirements verification data (30) can be understood as a checklist for the provision of a service.

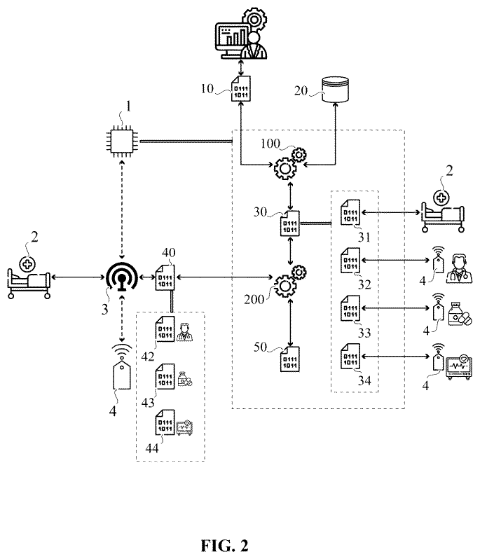

According to the above and FIG. 2, the requirements verification data (30) comprises a space allocation data (31) related to at least one service space (2); a staff allocation data (32) related to at least one electronic identification element (4); a supplies allocation data (33) related to at least one electronic identification element (4); and an equipment allocation data (34) related to at least one electronic identification element (4).

Optionally, the requirement verification data (30) may include a time allocation data (35), equivalent to the required time data (15), and a service allocation data (36), equivalent to the required service data (16). These data (35, 36) can be used to maintain traceability of the scheduling process (100) and serve to identify the allocation of resources and service area (2).

Additionally, in one embodiment of the present disclosure, the required time data (15) may be linked to the required personnel data (12) and the required service data (16). For example, in a modality in which the service area (2) is an operating room, where the required service data (16) may correspond to a procedure, and where the required personnel data (12) may correspond to a medical specialist related to an electronic identification element (4), the required time data (15) may correspond to the average time of the medical specialist to carry out the procedure, therefore, the time allocation data (35) is related to said average time.

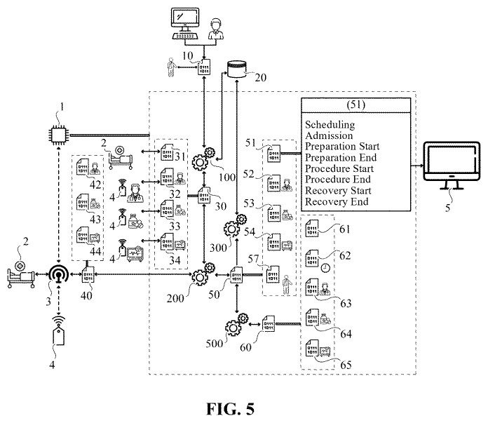

Optionally, and according to FIG. 5, the requirements verification data (30) may also include user assignment data (37) related to an electronic identification element (4). Said electronic identification element (4) can be assigned to a user of the service and activated at the time said user enters the service center.

In relation to the above, stage c), where the allocation of a service space (2) and resources is carried out according to a requirement data (10) and an availability data (20), allows to identify the needs for the provision of a service according to the capacity of a service center, which means less time and greater precision in the identification of resources compared to a method where a scheduling process is not carried out (100) based on the common characteristics between the requirement data (10) and the availability data (20).

In one embodiment of the present disclosure, the requirements verification data (30) is only generated when the conditions recorded in the requirements data (10) are satisfied by the availability recorded in the availability data (20). In other words, for each piece of data (11, 12, 13, 14) that includes the requirement data (10), there is a piece of data (21, 22, 23, 24), included in the availability data (20), that can be related by means of a scheduling process (100) to generate a requirement verification data (30).

In accordance with the above, if, despite the fact that the required space data (11) can be related to the available space data (21), the required personnel data (12) can be related to the available personnel data (22), and the required input data (13) can be related to the available input data (23), it is not possible to relate the required equipment data (14) with the available equipment data (24), for example, because the requested equipment is scheduled for maintenance or because the available equipment data (24) includes a greater number of rotations than the number of rotations that said equipment can have, it is not possible to generate a requirements verification data (30). According to the previous method, the process can be repeated from step a), meaning it is possible to create, modify, cancel, or delete a service provision.

Once the requirements verification data (30) has been generated in the computing unit (1), stage c) concludes and an interaction related to scheduling is considered completed, therefore, the service in the assigned attention space (2) can be provided.

After stage c) and during the provision of said service, in stage d) the identification data (40) is generated in the data acquisition device (3) by detecting an electronic identification element (4). Said identification data (40) corresponds to the entry and exit record to the attention space (2) of one or more resources associated with an electronic identification element (4).

In accordance with the above, stage d) allows for a precise identification of each of the objects (supplies and equipment) and people that enter a service space (2) during the provision of a service. This identification makes possible the real-time identification of the resources that may be needed immediately in a service space (2), generate an alert, or make a decision about the entry or exit of personnel and resources in said service space, which allows the reduction of waiting times related to the enlistment of a service space (2), given that there is a real-time identification of the resources that are in said service space (2).

Additionally, the data acquisition device (3) may comprise a communication module. Said communication module is linked to the computing unit (1), so that, when the data acquisition device (3) records an entry or an exit of an electronic identification element (4), said event can be recorded, thereby generating identification data (40), which can be transmitted to the computing unit (1).

For example, during step d), when a person with an electronic identification element (4) enters a service space (2) related to a data acquisition device (3), where said electronic identification element is related to a personnel assignment data (32), an identification data (40) is generated in the data acquisition device (3). Said identification data (40) can be transmitted to the computing unit (1) by means of a communication module.

According to the foregoing, with FIG. 2 and with FIG. 5, the identification data (40) comprises a personnel identification data (42) associated with at least one electronic identification element (4); a supplies identification data (43) associated with at least one electronic identification element (4); and an equipment identification data (44) associated with at least one electronic identification element (4).

To complete stage d), once the identification data is generated (40), and The data acquisition device (3) provides the computing unit (1) with the identification data (40) for the purpose of make a tracking and monitoring for inventory management and registration of the assisting staff during the provision of a service in the service area (2). The above allows processing of the input and output information of the electronic identification elements (4) in the computing unit (1).

In one embodiment of the present disclosure, step d) may be performed multiple times, in this way, multiple entries and exits of one or more electronic identification elements (4) related to a resource of the call center may be recorded.

In this way, after at least one iteration of stage d), stage e) is carried out, where the computing unit (1) is generated. the registration data (50) through the minimum requirements verification process (200). In this minimum requirements verification process (200), the requirements verification data (30) and the identification data (40) are compared and the input and output relationships are recorded. in the attention space (2) of one or more resources associated with one or more electronic identification elements (4).

According to the above, during stage e) the minimum requirements verification process (200) allows to identify the entry or absence of an element included in the requirements verification data (30). Such identification can be done at the time an element enters or leaves the attention space (2), or at any required time. In this way, said registration data (50) allows generate alerts related to the use of said service areas (appropriate use of the service area, completeness of required elements, entry of personnel, use of supplies).

In one embodiment of the present disclosure, before performing the minimum requirements verification process (200) of stage e), the following sub-stages can be performed for resource tracking:
record in the computing unit (1) the resources to be used according to availability and requirements verification data (30); and
associate a resource with an electronic identification element (4).

This allows for detailed monitoring of the resources used during the provision of a service.

For example, in a modality in which the care center is a health center, where the service area (2) is an operating room, and where the warehouse area verifies the availability of the supplies registered in the required supply data (13) to generate the available supply data (23), before the procedure, the warehouse area staff can identify and reserve some elements for the transportation of supplies, for example, transport baskets, associated with a scheduled procedure and related to an electronic identification element (4). After identifying and reserving said transport elements, the warehouse area staff can select some inputs, based on the required input data (13) and the available input data (23), and place said inputs inside the transport element related to an electronic identification element (4).

Such relationship between the transport element and the electronic identification element (4) may be recorded in the supply allocation data (33). According to the above, in stage c) when the transport element, which contains the selected inputs, is entered into the attention space (2), the identification data (40) is generated in the data acquisition device (3) by detecting the electronic identification element (4) related to the transport element.

In one embodiment of the present disclosure, the transport elements may not have an electronic identification element (4). In this case, according to one embodiment of the present disclosure, the transport element may be delivered to the service personnel related to an electronic identification element (4). According to the previous modality, during stage e), in the minimum requirements verification process (200) the entry of inputs is identified when service personnel enter related to an electronic identification element (4), and therefore, in the computing unit (1) a data record of inputs (53) is generated. The above allows to identify the personnel who transport the supplies and record their entry and exit from each service space (2), facilitating the monitoring of each supply.

In one embodiment of the present disclosure, where a transport element has been identified and reserved, and where said transport element has not been related to an electronic identification element (4), during the selection of inputs a record of inputs can be made, where each input is related to an electronic identification element (4). Under this modality, electronic registration allows for an automated inventory of the supplies related to a service center. The above is possible since the elements that may have returned to the warehouse area after the provision of a service can be identified more precisely, compared to a modality where each input is not related to an electronic identification element (4).

Optionally, the transport element may have a non-electronic identification element, for example, a code or a color assignment, which allows said transport element to be identified and related to the provision of a service.

Additionally, given the registration data (50) it allows to identify a residence time (ie time recorded between an entry and an exit) and a rotation time (ie number of times an entry or an exit is recorded) related to said electronic identification element (4).

According to the above and FIG. 5, the record data (50) may include a service status data (51), a supply record data (53), an equipment record data (54), and a service time record data (55).

In relation to the above, the service status data (51) corresponds to the result of a state machine executed during the minimum requirements process (200) of stage e), which is subsequent to stage d).

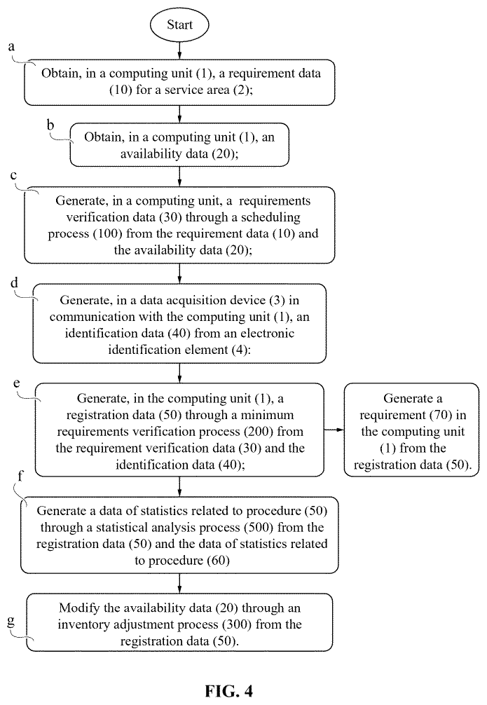

For example, in one embodiment of the present disclosure and according to FIG. 4, simultaneously with step e) it is possible to generate a requirement (70) from the registration data (50) during the minimum requirements process (200), where said requirement (70) is associated with the generation of an alert or action.

According to the above, the service status data (51) can be “Busy” when an identification data (40) is generated due to the presence of an electronic identification element (4) in the service space (2), where the personnel identification data (42) coincides with the personnel assignment data (32).

In another example, in step e) when the personnel identification data (42) does not match the personnel assignment data (32), the service status data (51) may correspond to “User does not correspond”'. Simultaneously, said state of “User does not correspond” indicates that a requirement (70) must be generated that corresponds to a visual or sound action in the attention space (2), for example, an alarm.

In accordance with the present disclosure, the state machine, the result of which corresponds to the service status data (51), can be configured according to the particular needs of the care center. In one embodiment of the present disclosure, the service status data (51) may be displayed on a display device (5) in a simultaneous or subsequent step to step e).

In one embodiment of the present disclosure, the service status data (51) is selected from the states of Scheduling, Admission, Start service, and Ends service. Additionally, said service data (51) can be displayed on a display device (5).

According to the previous modality, the service status data (51) corresponds to Scheduling when the data has been generated verification of requirements (30), that is, when between the requirement data (10) and the availability data (20) there has been established a relational bond.

In the same way, the service status data (51) corresponds to Admission when the data of verification of requirements (30) includes a user assignment data (37) related to an identification element (4), and said electronic identification element (4) is activated, that is, at the moment when said user enters the service center.

Likewise, the service status data (51) corresponds to Service begins when the identification element (4), related to the user assignment data (37), enters the attention space (2) and identification data (40) is generated in the data acquisition device (3), that is, when the user enters the attention space (2).

Similarly, the service status data (51) corresponds to Service ends when the identification element (4), related with the user assignment data (37), the user leaves the attention space (2) and identification data (40) is generated in the data acquisition device (3), that is, when the user leaves the attention space (2).

Additionally, the group of states corresponding to Service begins and Service ends can be directly related to the assigned attention space (2). Thus, if the attention space (2) corresponds, for example, to an operating room, the group of states Service begins and Service ends It can be Start surgery and End surgery.

With reference to FIG. 5, in an embodiment in which the space allocation data (31) corresponds to more than one attention space (2), the service status (51) obtained in step e) may include more than one group of states corresponding to the states Service begins and Service ends. For example, when the space allocation data (31) corresponds to a preparation room, an operating room and a recovery room, there may be three groups of states corresponding to Service begins and Service ends, for example, such groups of states may be, in relation to the preparation room Preparation begins, Preparation ends; in relation to the operating room Start procedure, Procedure ends; and in relation to the recovery room Recovery begins, and Recovery completes.

For its part, the personnel registration data (52) corresponds to a staff dwell time related with at least one electronic identification element (4) and associated with the personal identification data (42) that is calculated during the minimum requirements process (200). This data is personnel record (52) can be recorded individually, that is, for each electronic identification element (4), and corresponds to the personnel who participated during the provision of the service assigned to a service space (2).

In one embodiment of the present disclosure, the personnel record data (52) allows for modification the required time data (15) linked to the required personnel data (12) and the required service data (16).

On the other hand, the input registration data (53) corresponds with the balance resulting from a supply identification data (43A) corresponding to the inputs that enter the attention space (2), and a supply identification data (43B) that corresponds to the inputs that leave the attention space (2). Said supply identification data (43B) that corresponds to the inputs that leave the attention space (2) may not be the same as the supply identification data (43A) corresponding to the inputs that enter the attention space (2). In other words, the input registration data (53) corresponds to the inputs that were used during the provision of a service assigned to a service space (2), where, during the minimum requirements process (200) the supply identification data (43A) of input and the supply identification data (43A) of output are compared.

The equipment registration data (54) corresponds with the entry and exit record of one or more teams related with an electronic identification element (4) and associated with the equipment identification data (44). Said equipment registration data (54) corresponds to the time record of the equipment that was used during the provision of a service assigned to a service space (2) calculated through the minimum requirements process (200).

The attention time data (55) corresponds to the record of the time elapsed between the entry of a first identification element (4A) to the attention space (2), where a service is provided, and the exit of a last identification element (4B) from the attention space (2), where said identification elements (4A, 4B) may be different. In other words, the data on service time (55) corresponds to the total time that the provision of a service assigned to a service space (2) lasts, calculated in the minimum requirements process (200).

In this sense, the registration data (50) allows to identify the interactions between personnel, supplies and equipment in relation to a service space (2) for a service provided, that is, it allows to determine the activity times of the service personnel, a rate of use of the supplies, and a rate of use of the equipment during the provision of a service.

In one embodiment of the present disclosure, after step e), through an inventory adjustment process (300) and from the registration data (50) it is possible to modify the availability data (20). The above allows updating the interactions related to scheduling, for example, in case a service space (2) is not available as planned, it is possible to identify said change and reflect it in the availability data (20), in this way, carry out a new scheduling process (100). This process allows optimize the allocation and use of resources for a service.

Optionally, the registration data (50) comprises a service registration data (56), equivalent to the service assignment data (36); and a user data (57), equivalent to the user assignment data (37).

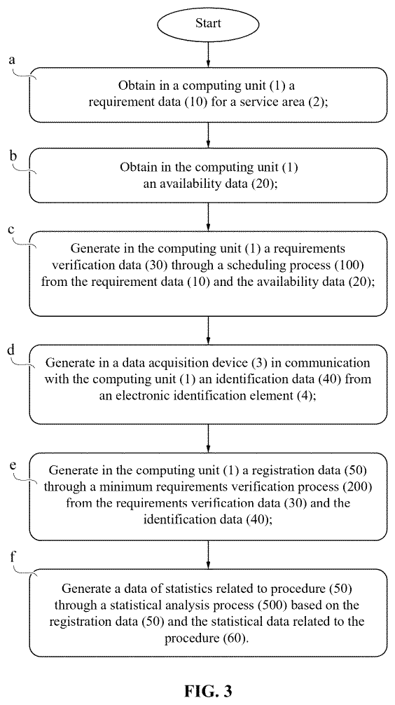

In one embodiment of the present disclosure, according to FIG. 3 and FIG. 5, after step e) and from the registration data (50), a statistical data related to the procedure (60) can be generated by means of a statistical analysis process (500). Said statistical analysis process (500) is a statistical type mathematical process.

In this sense, the statistical data related to procedure (60) includes historical information, as well as usage averages, occupancy averages, total expenses, and total service staff activity times, which which allows to obtain patterns as to to the behavior of the record data (50) stored historically. In this way, the statistical analysis process allows obtain a statistical analysis and management indicators related to a service area (2).

According to the above and FIG. 5, the data of procedure-related statistics (60) It includes data on the service provided (61), data on identified personnel (62), data on supplies used (63), data on equipment used (64), and data on time used (65).

In particular, the service provided data (61) is related to the required service data (25), where said service provided data (61) allows to exclusively identify the service that has been provided in a service space (2) of the service center.

On the other hand, the data of identified personnel (62) allows to characterize the personnel who have been present in a service area (2) during the provision of one or more services, as well as the time of permanence and rotation of said personnel in a service area (2).

Similarly, the data of inputs used (63) and the data of equipment used (64) allow to identify, respectively, the inputs and equipment that have entered and that have been used during the provision of one or more services in a service area (2), where said inputs and equipment are related to an electronic identification element (4). In the particular case of the data on equipment used (64), this data also allows us to know the total time of use of said equipment.

For its part, the data on time used (65) is related to the record of income and expenditure of resources associated with a service provided in a service area (2). In this way, the time data used refers to the set of records of The data on attention time (55). In other words, the time used data (65) corresponds to the total usage times that have been recorded during the provision of one or more services assigned to a service space (2).

Additionally, prior to the provision of the service, a verification and preparation of the elements corresponding to each of the electronic identification elements (4) related to the requirements verification data (30) can be carried out. For example, with reference to the supply allocation data (33), a preparation can be carried out prior to the start of the service provision. In said preparation, for example, the elements that correspond to each identification element (4) related to the supply allocation data (33) can be physically arranged, in such a way that they can be transferred from a storage location to the service space (2).

According to the present disclosure, steps a) to e) can be implemented in a system for the logistics management of spaces comprising a computing unit (1), a data acquisition device (3) in communication with a computing unit (1), and at least one electronic identification element (4) related to a resource or a user.

In particular, said data acquisition device (3) is related to a service area (2). Said data acquisition device (3) allows information to be collected from an electronic identification element (4) related to a resource, in this way, it is possible to distinguish and exclusively recognize said resource within a larger set of independent resources.

In accordance with the above, the method of this disclosure allows linking the information of each procedure from the monitoring of supplies, equipment, personnel and patients. This is achieved through the use of electronic identification elements (4) related to the resources associated with said care center, and data acquisition devices (3) related to a service area (2).

Accordingly, by way of example, in one embodiment of this disclosure the care center may be a medical care center. In this way, the service area (2) can refer to a preparation room, a recovery room, an operating room, among others, where said service area (2) is related to a data acquisition device (3).

For its part, said health care center has available resources, said resources can be identified as personnel (e.g. cleaning staff, warehouse staff, doctors, administrative staff), supplies (e.g. medications, sterilization material, protection and safety materials, support materials), and equipment (e.g. surgical table, lamps, stretchers, suction systems, monitoring systems, specialized instruments), where said resources are related to at least one element of electronic identification (4).

The aforementioned system allows for reducing wait times related to the preparation of a service space, generating alerts related to the use of said service spaces (proper use of the service space, completeness of required elements, staff entry, use of supplies), optimizing the allocation and use of resources for a service, and obtaining statistical analysis and management indicators related to each space.

### EXAMPLES

### Example 1

In a medical center, the support staff schedules a new service for a service area (2), where the service is a medical procedure and the service area (2) includes a preparation room, an operating room, and a recovery room.

The process of scheduling a new service included the following steps:
a) A data requirement (10) for the preparation room, the operating room, and the recovery room was obtained in a computing unit (1).

This requirement data (10) is defined in the following table:

| required space | required personnel | required input | required equipment | required time | required service |
|---|---|---|---|---|---|
| data (11) | data (12) | data (13) | data (14) | data (15) | data (16) |
| Preparation | P. service | Coat | Stretcher | 10 min. | Preparation |
| room | Nursing | | | | |
| Operating | P. service | Alcohol | Surgery | 2 Hours | Appendectomy |
| room | Anesthesiology | Gauze | table | | |
| | Surgery | | Vital signs | | |
| | Nursing | | monitor | | |
| Recovery | P. service | | Stretcher | 2 Hours | Recovery |
| room | Nurse | | Vital signs | | |
| | | | monitor | | |

b) availability data (20) was obtained in the computing unit (1). This availability data (20) corresponds to the inventory of supplies, the inventory of equipment, and the hours of attention of the staff at the medical center;
c) A requirement verification data (30) was generated in the computing unit (1) through a scheduling process (100).

Said requirements verification data (30) is defined in the following table:

| space allocation | staff allocation | supply allocation | equipment assignment | time allocation |
|---|---|---|---|---|
| data (31) | data (32) | data (33) | data (34) | data (35) |
| Preparation | S. general 1020 | Bata (OK) | Camilla (OK) | 2:00 pm |
| Room 303A | S. general 1025 | | | |
| | Nurse 5463 | | | |
| Operating | S. general 1020 | Alcohol (OK) | Surgery table | 2:10 pm |
| Room 205C | S. general 1025 | Gauze (OK) | (OK) | |
| | Anesthesiologist | | Vital signs | |
| | 6987 | | monitor (OK) | |
| | Surgeon 98163 | | | |
| | Nurse 5463 | | | |
| Recovery Room | S. general 1020 | | Camilla (OK) | 5:10 pm |
| 103D | S. general 1025 | | Vital signs | |
| | Nurse 5463 | | monitor (OK) | |

At the end of stage c), the interaction related to scheduling was considered complete, therefore, the service in the assigned service space (2) could be provided.

The admissions staff registered the patients on the day of the procedure, assigning an identification element (4) related to a user assignment data (37). Said electronic identification element (4) is an RFID or RTLS Tag, which was activated at the time said user entered the service center.

The patient, staff and resources admission were recorded in the following stage:
d) An identification data (40) was generated in a data acquisition device (3), consisting of an RFID antenna or router, in communication with the computing unit (1) from each electronic identification element (4) that enters the assigned service space (2);
e) A record data (50) was generated in the computing unit (1) through a verification process of minimum requirements (200).

The patient, carrying the RFID or RTLS tag, entered preparation room 303A at 2:03 pm on a stretcher. The tag was read by the RFID antenna or router, which recorded the time of entry into the area.

The service status data (51), when entered by the user, is equivalent to the status “STARTS PREPARATION”, according to the code associated with the patient. This data was shown on a display device (5).

The patient, carrying the RFID or RTLS tag, left preparation room 303A at 2:15 p.m. The Tag is read by the RFID antenna or router that records the time of stay, and the service status data (51), changed to the “PREPARATION ENDS” status, according to the code associated with the patient. This data was shown on a display device (5).

The patient, carrying the RFID or RTLS Tag, entered operating room 206C at 2:17 pm on a stretcher and the Tag is read by the RFID antenna or router. A “NO OPERATING ROOM CORRESPONDENCE” alert was generated indicating the error.

The patient, carrying the RFID or RTLS Tag, entered operating room 205C at 2:19 pm on a stretcher and the Tag is read by the RFID antenna or router.

The RFID antenna or router recognizes the Tags associated with the equipment. The support staff enters and the RFID antenna or router recognizes the associated Tags. The staff in charge records the supplies using a barcode. The service status data (51) changed to the “SURGERY STARTS” status, according to the code associated with the patient.

According to the previous example, the entry of the patient, equipment, supplies, and staff attending a surgery was recorded, and an alert was generated when the patient entered a space that did not correspond to the one assigned.

### Example 2

In a medical center, the surgical scheduling staff generates an appointment for a service for a service area (2), where the service is a cardiac pacemaker implantation and the service area (2) includes a preparation room, a radiation-shielded operating room, and a recovery room.

The process of scheduling a new service was carried out in the following stages:
a) A data requirement (10) for the preparation room, the operating room, and the recovery room was obtained in a computing unit (1).

This requirement data (10) is defined in the following table:

| required space | required personnel | required input | required equipment | required time | required service |
|---|---|---|---|---|---|
| data (11) | data (12) | data (13) | data (14) | data (15) | data (16) |
| Preparation | P. service | | | 2 Hours | Preparation |
| room | Nursing | | | | |
| Operating | P. service | Alcohol | Anesthesia | 1 hour. | Cardiac |
| room with | Circulating | Suture | machine | | pacemaker |
| radiation | Instrumentalist | needles. | C-arch | | implantation |
| protection | Physician | Surgical | Vital signs | | |
| | Assistant | staples. | monitor | | |
| | Anesthesiologist | Clamps and | Surgical table | | |
| | Surgeon | occluders for | Electrosurgery | | |
| | Nursing | organs and | | | |
| | | blood vessels. | | | |
| | | Dilators. | | | |
| | | Gauze. | | | |
| | | Syringes. | | | |
| | | Bandages. | | | |
| | | Sterile | | | |
| | | gloves. | | | |
| | | Catheters | | | |
| | | and urine | | | |
| | | bags. | | | |
| Recovery | P. service | | | 2 Hours | Recovery |
| room | Nursing | | | | |

In order to verify the availability of resources to obtain availability data (20), the staff of the surgery scheduling area, after generating the appointment, carried out a verification process with the insurer to confirm the provision of the service.

After verification with the insurer, the following step was taken:
b) The availability data (20) was obtained in the computing unit (1). This availability data (20) corresponds to the inventory of supplies, the inventory of equipment, and the hours of attention of the staff at the medical center.

The staff in the surgery scheduling area verified the availability of operating rooms according to the requirements recorded in the required space data (11). Based on this availability, a data of available space (21) was generated, indicating that preparation room 304A, operating room 1, and recovery room 104A were available.

In addition, the surgical scheduling staff verified the availability of the specialists required for the surgery. According to this availability, data on available personnel was generated (22), indicating that service personnel 1022 and 1015, nursing staff 1241 and 1248, circulating staff 1546, instrument technician 5123, assistant physician 4265,anesthesiologist 3698, and surgeon 6845 were available to provide the service.

Similarly, warehouse staff verified the availability of the supplies required for the surgery and linked the baskets to the scheduled procedure. The warehouse staff was responsible for assigning an identifier to each procedure.

The warehouse area staff, one day before the scheduled procedure, carried out the selection of inputs by procedure using ESL (electronic paper) and barcode, with this, generating available input data (23).

On the other hand, the biomedical area staff verified the availability of the equipment for the scheduled appointment and generated the reservation of the requested equipment, which was available, generating data on available equipment (24). Additionally, the biomedical staff updated the equipment's resumes.

A requirements verification data (30) was generated in the computing unit (1) according to the following stage:
c) A requirement verification data (30) was generated in the computing unit (1) through a scheduling process (100). Said requirements verification data (30) is defined in the following table:

| space allocation | staff allocation | supply allocation | equipment assignment | time allocation |
|---|---|---|---|---|
| data (31) | data (32) | data (33) | data (34) | data (35) |
| Preparation | P. service 1022 | | | 2 Hours |
| Room 304A | Infirmary 1241 | | | |
| Operating | P. service 1022 | Alcohol (OK) | Anesthesia | 1 hour. |
| Room 1 | Circulating 1546 | Suture needles | machine (OK) | |
| | Instrumentation | (OK) | C-arm (OK) | |
| | Technician 5123 | Surgical staples | Vital signs | |
| | Physician Assistant | (OK) | monitor (OK) | |
| | 4265 | Clamps and | Operating | |
| | Anesthesiologist | occluders for | table (OK) | |
| | 3698 | organs and blood | Electrosurgery | |
| | Surgeon 6845 | vessels (OK) | (OK) | |
| | Infirmary 1248 | Dilators (OK) | | |
| | | Gauze (OK) | | |
| | | Syringes (OK) | | |
| | | Bandages (OK) | | |
| | | Sterile gloves | | |
| | | (OK) | | |
| | | Catheters and | | |
| | | urine bags (OK) | | |
| Recovery Room | P. service 1015 | | | 2 Hours |
| 104A | Infirmary 1241 | | | |

Before the procedure, the warehouse staff reserved a basket for the supplies. This basket was identified by means of a color.

The day before the surgery, the warehouse staff kept a record of supplies, linking each supply to a QR code. Recording each input in a database. This allowed for the preparation of a report of the elements that had been dispatched for the surgery.

The admissions staff registered the patients on the day of the procedure, assigning an identification element (4) related to a user assignment data (37). Said electronic identification element (4) is an RFID or RTLS Tag, which was activated at the time said user entered the service center.

On the day of the procedure, warehouse staff verified the contents of the baskets before they were delivered for each confirmed procedure, using the QR code for each item. Additionally, the basket was assigned to the 1546 circulating staff, who were responsible for delivering the baskets to each operating room according to each confirmed procedure.

The patient, staff and resources admission were recorded in the following stage:
d) An identification data (40) was generated in a data acquisition device (3), consisting of an RFID antenna or router, in communication with the computing unit (1) from each electronic identification element (4) that enters the assigned service space (2).

The entry and exit record of each electronic identification element was carried out in the following stage:
e) A record data (50) was generated in the computing unit (1) through a verification process of minimum requirements (200).

The patient, carrying the RFID or RTLS tag, entered preparation room 304A. The service status data (51), when entered by the user, is equivalent to the status “STARTS PREPARATION”, according to the code associated with the patient. This data was shown on a display device (5).

Circulating staff 1546 entered preparation room 304A and delivered the basket with the supplies. The Tag is read by the RFID antenna or router that records the entry of circulating personnel 1546 and the delivery of supplies.

The patient left the preparation room, carrying the RFID or RTLS tag. The Tag is read by the RFID antenna or router that records the time of stay, and the service status data (51), changed to the “PREPARATION ENDS” status, according to the code associated with the patient. This data was shown on a display device (5). The patient spent 1.5 hours in the preparation room.

The admission to operating room 1 of 5123 instrument technicians, 3698 anesthesiologists, 1248 nursing staff, and 4265 assistant physicians was recorded.

After 10 minutes, the entry of surgeon 6845 was not registered, a request (70) was generated to make an urgent call to surgeon 6845.

The admission to operating room 1 of surgeon 6845 was recorded.

The patient, carrying the RFID or RTLS tag, entered operating room 1. The service status data (51) changed to the “SURGERY STARTS” status, according to the code associated with the patient.

The RFID antenna or router recognizes the Tags associated with the equipment. The support staff enters and the RFID antenna or router recognizes the associated Tags.

The scheduled procedure is performed.

The patient, carrying the RFID or RTLS tag, left the operating room. The Tag is read by the RFID antenna or router that records the time of stay, and the service status data (51), changed to the “SURGERY ENDS” status, according to the code associated with the patient. This data was shown on a display device (5). The patient was in the operating room for 3 hours.

At the end of the surgery, a request was generated (70) to make a call to the cleaning staff 1022, said call was made through a web application. Additionally, said call was shown on a display device (5).

Additionally, a time count was started from the end of the surgery until service personnel 1022 arrived to clean operating room 1. Likewise, the system automatically notified the cleaning staff via a web application, prompting them to proceed with cleaning the operating room.

Service personnel 1022 entered operating room 1 and performed a manual check-in to begin counting the cleaning time. This data was shown on a display device (5).

Once the cleaning process was completed, service personnel 1022 performed a manual log to complete the cleaning time count for Operating Room 1. This data was shown on a display device (5).

Likewise, once the procedure was completed, circulating personnel 1546 returned the supplies to the warehouse area.

The warehouse staff calculated the shortages using an automatic count using RFID.

The warehouse staff uploaded the expense report to the system, updated the inventory, and detached the inputs associated with the procedure. In accordance with the above, After stage e), through an inventory adjustment process (300) and from the registration data (50), the availability data (20) was modified, where the inputs used and the inputs returned to the warehouse area were recorded.

According to the previous example, the entry of the patient, equipment, supplies, and staff attending a surgery was recorded, and an alert was generated when the patient entered a space that did not correspond to the one assigned.

Additionally, the patient's workflow was recorded, tailored to the particular workflow of the care center, where the different workflows depend on whether the procedure is outpatient, emergency, isolated, ICU, or inpatient.

Using the RFID antenna or router, the following times were recorded:
1. Preparation begins
2. Preparation ends
3. Patient admission to the operating room
4. Patient leaving the operating room
5. Recovery Begins
6. Recovery completes

Likewise, the following times were recorded in operating rooms:
1. Time to start surgery
2. Time starts anesthesia
3. Time ends anesthesia
4. Surgery completion time
5. Once the surgery is over, the cleaning staff begins to count the time it takes to arrive at the operating room to begin the cleaning process.
6. Cleaning start time
7. Cleaning end time

### Example 3

Scheduling and service provision were carried out in accordance with Example 2. After stage e), from the registration data (50), a statistical data related to the procedure (60) was generated through a statistical analysis process (500).

From the data of time used (65) and registration (50) the following information was collected:
1) Surgeries by specialty: Total number of surgical procedures, calculating their weekly frequency and annual distribution; also classified by specialty.
2) Compliance with the scheduled time: Indicator of compliance with scheduled surgical procedures, where the percentage by week and distribution by year are calculated.
3) Omissions and Cancellations to the surgical program: It is the ratio of the total number of canceled and omitted surgeries in the week to the number of surgeries scheduled for that same week.
4) Omissions and cancellations for reasons: Percentage of surgeries canceled during a week for reasons not due to the patient.
5) Occupancy percentage: Percentage of operating room utilization in relation to their regularly available hours.
6) Percentage of emergencies: Number of surgical procedures performed urgently.
7) Delay Generated: Cumulative delay time for all procedures during the filtered week
8) Compliance with the Standard: Comparison of the scheduled duration of the procedure and the time executed, if the value is negative it indicates that the procedure lasted less than expected.

### GLOSSARY

Below are some of the terms used in some modalities of the method disclosed throughout the Descriptive Chapter:
Service: A service is understood to be an activity or set of activities aimed at providing care, assistance, treatment, intervention, or support to a user who requires specialized care.

User A user is understood to be an entity or object to which a service is directed. In this sense, a user refers to a person, animal, or object that actively or passively uses or benefits from a service and takes part in the activities required to complete that service.

For example, in the field of health services provision, the user may be a patient who is about to undergo a medical procedure.

Another example, in the field of automotive service, the user may be a vehicle on which a mechanical procedure is to be performed.
Attention space: A service space is understood to be a physical area, environment, or place intended to provide a specialized service to a user.

In a care setting, the goal is to provide a favorable environment and appropriate means for interaction and the provision of said service. This may involve the provision of equipment, furniture, security systems, and other aspects that contribute to the user's well-being.

This service area may vary depending on the type of service offered; for example, in the medical field, a service area may refer to an operating room, an office, a preparation room, a recovery room, a diagnostic laboratory, etc.
Center of attention: A service center is understood to be a physical area or environment where the provision of one or more specialized services is facilitated.

According to the above, a service center may be associated with more than one service space, where each of said service spaces may be dedicated to providing a specific service. For example, a hospital may be a care center that includes various service areas, such as operating rooms, intensive care units, consulting rooms, etc.

RESOURCES: A resource shall be understood as the elements, assets or means that are available to be used in order to provide a service.

In the context of this disclosure, resources are associated with the care center and may be allocated to provide a specific specialized service in a service area.

According to the above, it will be understood that a care center includes resources related to service areas (physical plant), personnel (human resources), and equipment and supplies (material resources or inventory).
Staff: The term “staff” shall be understood to refer to individuals who perform a service provision function. For example, in the context of a healthcare facility, staff may include general service providers (e.g., cleaning staff, warehouse staff), healthcare service providers (e.g., physicians), and administrative service providers (e.g., clerical staff).
Input: The term “input” shall be understood to refer to an element, product or device that is consumable or has a short life cycle. This implies that the item is used only once or for a limited period before being discarded.

For example, in the medical field, a consumable item can be a medicine or a chemical substance.
Equipment: The term “equipment” shall be understood to refer to an item that is not consumable, meaning that, unlike a consumable item, it is not used on a single or disposable basis and may have a longer useful life.

Such non-consumable items may be essential for the provision of a service, for example, in the healthcare field, non-consumable items may refer to devices or equipment that can be used repeatedly after being sterilized or disinfected (e.g. monitoring equipment, diagnostic imaging equipment, life support equipment).
Computing unit: Said control unit (13) can be selected from the group consisting of: programmable logic controllers (PLC), microprocessors, DSCs (Digital Signal Controller), FPGAs (Field Programmable Gate Array), CPLDs (Complex Programmable Logic Device), ASICs (Application Specific Integrated Circuit), SoCs (System on Chip), PsoCs (Programmable System on Chip), computers, servers, tablets, cell phones, smart phones, signal generators and equivalent control units known to a person of ordinary skill in the art and combinations thereof.

The computing unit may include a storage device, a display device, an output device, a Human Interface Device (HID), a communications module. Furthermore, the computing unit may be or include a special purpose computing unit programmed to execute the method of this disclosure.
Remote Device: A remote device will be understood as a device with a computing unit that is allowed to share information with the computing unit (1) and that occupies a different physical space than the latter.
Communication module: A communication module is understood to be a device or system that allows the sending and receiving of information to and from the computing unit from an external device. Said communications module may be a wired communications module, a wireless communications module, or a wired and wireless communications module.

Said control unit (13) and the second control unit (13b) may be connected by a wireless communications module, which uses a wireless communication technology that is selected from the group consisting of Bluetooth, WiFi, RF ID (Radio Frequency Identification), UWB (Ultra Wide Band), GPRS, Konnex or KNX, DMX (Digital MultipleX), WiMax, and equivalent wireless communication technologies known to a person of ordinary skill in the art, and combinations thereof.

For its part, the wired communication module has a wired connection port that allows communication with external devices through a communications bus, which is selected, among others, from the group consisting of I2C (from the acronym for IIC Inter-Integrated Circuit), CAN (for the acronym in English for Controller Area Network), Ethernet, SPI (for the acronym in English for Serial Peripheral Interface), SCI (for the acronym in English for Serial Communication Interface), QSPI (for the acronym in English for Quad Serial Peripheral Interface), 1-Wire, D2B (for the acronym in English for Domestic Digital Bus), Profibus and others known to a person moderately versed in the subject.
Display device: A computing unit shall be understood as a device that can be connected to a computing unit and display its output, selected from among others a CRT monitor (Cathode Ray Tube), flat panel display, LCD liquid crystal display (Liquid Crystal Display), active matrix LCD display, passive matrix LCD display, LED displays, display projectors, TV (4KTV, HDTV, plasma TV, Smart TV), OLED displays (Organic Light Emitting Diode), AMOLED displays (Active Matrix Organic Light Emitting Diode), QD quantum dot displays (Quantum Display), segment displays, among other devices capable of displaying data to a user, known to those skilled in the art, and combinations thereof.
Data acquisition device: A data acquisition device is understood to be a piece of equipment or system designed to collect information from one or more external sources and convert it into data that can be directly interpreted or transmitted to a computing unit for subsequent interpretation or storage.

In the context of the present disclosure, the data acquisition device allows the recording of the location or presence of an identified element within a specific space, said identification can be carried out by specific characteristics of the element (detection by sensors), optical detection, electronic detection, manual recording, biometric recording, or any type of detection and recording known to a person moderately versed in the matter.

Similarly, said data acquisition device communicates with the computing unit by means of a communication module.
Electronic identification element: An electronic identification element is understood to be an object that incorporates technology that allows the identity of a person or object to be authenticated and verified by electronic means.

According to the above, the electronic identification element (4) is an object that incorporates a technology that is selected from among bar code, QR code (Quick Response Code), RFID (Radio Frequency Identification Device), RTLS (Real Time Location Systems), Bluetooth, BLE (Bluetooth Low Energy), and technologies known to a person moderately versed in the subject.
Fact: In the context of this disclosure, data shall be understood as a symbolic representation of an entity, said representation may be information encoded in numerical, alphabetical, alphanumeric, vector and/or matrix form.

According to the above, a piece of data can be represented by at least one bit of information. In this sense, a piece of data can have a structure, or frame, composed of blocks of characters, or bits, that represent different types of information. Each block can form strings of characters, numbers, logical symbols, among others. Different strings can configure a matrix of n rows by m columns.

According to the above, data can be formed only of bits (strings in binary language), formed of characters organized one by one by a combination of bits, formed from fields, records, tables, or any structured representation of information. In particular, a piece of data can also be made up of several pieces of data.

ProcessA process is understood as a set of logical actions, which can be defined by means of a sequence of steps. These ducks are implemented on one or more data, where said data is manipulated, transformed, operated or processed, to generate new data.

According to the above, a process requires that there be a data input and a data output. A process can have one input and one output, multiple inputs and one output, or multiple inputs and multiple outputs.
Scheduling process: A scheduling process is understood as a process between two or more input data, for example, a relational process, where said data are interconnected or related to each other in some significant way. These relationships can be of different types and involve interactions, influences, dependencies or connections between the data involved.

According to the above, the resulting data is formed from the data that has satisfied these restrictions and allows to guarantee the referential integrity between the supplied data.

Specifically, in the field of surgical scheduling, the scheduling process is the process by which a hospital institution allocates the resources it has to meet the demand for surgical interventions. The resources or variables that comprise it include the spaces and time available, the duration of the surgeries, the necessary supplies, and the availability of medical resources.
Minimum requirements verification process: A minimum requirements verification process will be understood as a process between two or more data that represent one or more elements, and where said data are compared for the purpose of recording the relationships of presence and absence of an element.
Statistical analysis process: A statistical analysis process is understood to be a process in which one or more input data are systematically stored in a memory record with the objective of applying statistical techniques and analytical methods to explore, describe and analyze said data.

Such statistical techniques may include calculations of summary measures (such as mean, median, standard deviation), hypothesis testing, regression analysis, analysis of variance, among others.

In particular, the use of data analysis methods to monitor factors and indicators in services is key in the surgical field to improve processes and service delivery.

Additionally, the application of statistical analysis and process control in healthcare services in organizations serves to detect anomalies and propose standards in processes and services, with the goal of implementing standards that lead to good healthcare practices and optimize service delivery processes.

According to the above, the analysis of indicators serves to generate planning and predictability strategies for the improvement of services and processes.

It is to be understood that the present invention is not limited to the embodiments described and illustrated herein, for as will be evident to a person of ordinary skill in the art, there are possible variations and modifications that do not depart from the spirit of the invention, which is only defined by the following claims.

1. A method for the logistics management of a service area, comprising the stages of:
a) obtain in a computing unit (1) a requirement data (10) for a service area (2);
b) obtain in the computing unit (1) an availability data (20);
c) generate in the computing unit (1) a requirements verification data (30) through a scheduling process (100) from the requirement data (10) and the availability data (20), wherein the requirements verification data (30) comprises:
a space allocation data (31) related to at least one service area (2);
a personnel assignment data (32) related to at least one electronic identification element (4);
an supply allocation data (33) related to at least one electronic identification element (4); and
an equipment allocation data (34) related to at least one electronic identification element (4);
d) generate in a data acquisition device (3) in communication with the computing unit (1) an identification data (40) from an electronic identification element (4), wherein the identification data (40) comprises:
a personal identification data (42);
a supply identification data (43); and
an equipment identification data (44);
e) generate in the computing unit (1) a registration data (50) through a minimum requirements verification process (200) from the requirements verification data (30) and the identification data (40);
wherein the data acquisition device (3) is related to at least one service area (2).

2. The method of claim 1, wherein the service area (2) is a surgical service area.

3. The method of claim 1, wherein the registration data (50) comprises:
a service status data (51);
a programming time data (52);
a data on attention time (53); and
a supply usage data (54).

4. The method of claim 3, further comprising a step of generating a data of statistics related to procedure (60) through a statistical analysis process (500) based on the registration data (50) and the statistical data related to the procedure (60).

5. The method of claim 4, wherein the statistical data related to procedure (60) comprises:
a performed procedure data (61);
a time used data (62);
an identified personnel data (63);
a used supplies data (64); and
a used equipment data (65).

6. The method of claim 3, further comprising a step of modifying the availability data (20) by means of an inventory adjustment process (300) from the registration data (50).

7. The method of claim 3, further comprising a step of generating a request (70) in the computing unit (1) from the registration data (50).

8. The method of claim 3, wherein the registration data (50) comprises a user data (57).

9. The method of claim 8, wherein the service status data (51) is selected from Scheduling, Admission, Preparation Start, Preparation End, Procedure Start, Procedure End, Recovery Start, and Recovery End.

10. The method of claim 9, further comprising a step of displaying the service status data (51) on a display device (5).
